(19) United States
(12) Patent Application Publication
(10) Pub. No.: US 2026/0179500 A1
(43) Pub. Date: Jun. 25, 2026

(54) SYSTEM AND METHOD FOR CREATING PERSONALIZED CONTENT TO FACILITATE A BEHAVIORAL AND EMOTIONAL TRANSFORMATION IN A USER

(71) Applicant: Jacob Martin, Tucson, AZ (US)

(72) Inventor: Jacob Martin, Tucson, AZ (US)

(21) Appl. No.: 19/429,530

(22) Filed: Dec. 22, 2025

Related U.S. Application Data
(60) Provisional application No. 63/737,818, filed on Dec. 23, 2024.

(51) Int. Cl. G09B 5/02; A61B 5/00; G09B 5/04; G09B 5/06

(52) U.S. Cl. CPC G09B 5/02; A61B 5/742; A61B 5/7475; G09B 5/04; G09B 5/065

(57) ABSTRACT

The invention discloses a system and method for creating personalized content to facilitate a behavioral and emotional transformation in a user. The system includes a user state analyzer module that collects and analyzes data regarding a user's current state and desired state. At least one gap is identified between the user's current state and desired state. A content generation module dynamically creates personalized interventions aimed at bridging the identified gap. These interventions are delivered to the user through a delivery module, which presents the interventions in formats such as audio, video, and/or immersive virtual reality (VR).

### CROSS REFERENCE TO RELATED APPLICATIONS

This application claims benefit from currently pending U.S. Provisional Application No. 63/737,818 titled “A system and method for creating personalized content to facilitate a behavioral and emotional transformation in a user” and having a filing date of Dec. 23, 2024, all of which is incorporated by reference herein.

### FIELD OF THE INVENTION

The present technology pertains to systems and methods in the field of personalized content creation utilizing artificial intelligence (AI) and neuroscience principles and even more specially facilitating behavioral and emotional transformation through the dynamic generation of audio, video, and virtual reality (VR) content, tailored to individual user needs and progress in personal development and self-improvement applications.

### BACKGROUND OF THE INVENTION

The human mind is a complex entity, capable of experiencing a wide range of emotions and behaviors. These emotions and behaviors can be influenced by various factors, including personal experiences, environmental stimuli, and physiological conditions. In some instances, individuals may desire to change certain aspects of their emotional or behavioral state, such as reducing stress, improving focus, or enhancing emotional resilience. Internal conflicts can inhibit behavioral change, including misalignment between conscious intention and learned behavioral conditioning.

At the core of the human condition is a deep misalignment between the conscious and unconscious mind. While we consciously set goals and desire growth, the unconscious, which drives over 90% of our behavior, operates from past experiences, emotional associations, and identity patterns we didn't consciously choose. This split leads to self-sabotage, hesitation, and inconsistency, and it cannot be solved with logic or willpower alone.

This fundamental discord manifests in countless ways throughout our daily lives, creating a persistent sense of internal friction that many people struggle to understand or articulate. Our conscious mind operates like a skilled architect drawing blueprints for the life we want to build. It sets ambitious goals, creates detailed plans, and generates genuine enthusiasm for positive change. Yet this same conscious mind represents only the tip of an enormous psychological iceberg, with most mental processes occurring beneath the surface of awareness.

The unconscious mind, in contrast, functions more like an ancient security system that has been programmed by every significant experience we've ever had. It stores not just memories, but the emotional charge associated with those memories, the conclusions we drew about ourselves and the world, and the survival strategies we developed in response to various challenges and traumas. These unconscious programs run continuously in the background, influencing our perceptions, reactions, and choices in ways we rarely recognize. They operate with a different logic entirely, one that prioritizes emotional safety and familiar patterns over conscious intentions or rational goals.

This creates a kind of internal warfare where our conscious desires frequently clash with unconscious resistance. We might consciously decide to pursue a healthier lifestyle, but find ourselves mysteriously drawn to old habits when stress arrives. We may genuinely want to build closer relationships, yet discover ourselves withdrawing or creating conflict when intimacy becomes available. We set professional goals with sincere motivation, only to procrastinate, second-guess ourselves, or find excuses to avoid the very opportunities we claim to want. These patterns emerge not from laziness or lack of discipline, but from unconscious protective mechanisms that view change itself as potentially dangerous. The unconscious doesn't distinguish between physical survival and psychological comfort, so it may sabotage a career change that could lead to greater satisfaction if that change triggers old fears of abandonment, failure, or inadequacy.

What makes this dynamic particularly challenging is that traditional approaches to personal development often attempt to override unconscious resistance through sheer force of will. We try to think our way out of emotional patterns, use positive affirmations to counteract negative beliefs, or employ discipline to push through internal resistance. While these methods may produce temporary results, they rarely create lasting change because they fail to address the underlying unconscious programming that generates the unwanted patterns in the first place. It's like trying to change the course of a river by placing stones on the surface while ignoring the deeper currents beneath.

The unconscious mind also operates through what might be called “identity protection,” maintaining a consistent sense of self even when that identity includes limiting beliefs or self-destructive patterns. If someone's unconscious identity includes the belief that they are unworthy of success, they may unconsciously sabotage opportunities that would challenge this core belief. The mind finds it less threatening to remain consistent with a negative self-image than to risk the psychological reorganization that would come with genuine change. This explains why intelligent, capable people often struggle with seemingly simple changes, why success can feel uncomfortable or scary, and why we sometimes find ourselves acting in ways that contradict our conscious values and intentions.

Furthermore, this misalignment creates a cascade of secondary problems that compound the original split. The frustration of repeatedly failing to follow through on conscious intentions can lead to self-criticism, shame, and a sense of personal inadequacy. We begin to distrust our own commitments and may develop learned helplessness about our capacity for change. The energy required to constantly battle internal resistance becomes exhausting, leading to decision fatigue and emotional depletion. Over time, many people simply lower their expectations or abandon their deeper aspirations altogether, settling for a life that feels manageable but ultimately unfulfilling.

Resolution of this fundamental misalignment requires approaches that acknowledge and work with the unconscious mind rather than against it. This involves developing awareness of unconscious patterns, understanding the historical origins of resistance, and finding ways to create safety for the unconscious while pursuing conscious goals. The goal is not to eliminate the unconscious mind's protective function, but to update its programming so that it supports rather than sabotages our conscious intentions, creating an internal alliance that makes sustainable change possible.

Current technologies in content creation for personal growth often fall short in delivering personalized and contextually relevant guidance. The static nature of traditional approaches does not accommodate the dynamic, evolving journey of personal development that many individuals experience. Additionally, such technologies lack comprehensive feedback mechanisms to refine interventions based on user engagement or progress, resulting in limited efficacy in sustaining long-term behavioral changes or emotional transformations.

Furthermore, while audio, video, and virtual reality technologies have seen substantial advancements, their application in the personal development sector remains underexplored. Existing solutions do not fully exploit the potential of these modalities to create immersive and multisensory environments, which could significantly enhance engagement and retention of positive behavioral changes.

What is needed is a system that leverages advanced artificial intelligence to conduct dynamic user state analyses and create personalized interventions tailored to the individual's evolving needs.

### SUMMARY OF THE INVENTION

The present invention provides a system and method for creating personalized content to facilitate a behavioral and emotional transformation in a user. The system includes an interface with both input capabilities and a presentation component, along with at least one memory storage that contains instructions for at least one processor to execute. The interface is designed to guide either the user directly or a proxy acting on their behalf to enter relevant information. Based on this entered information, the system defines the context of the user's situation, their current state, their desired state, and calculates the gap that exists between the current and desired states.

Using this gap analysis, the system selects one core rewiring action along with multiple complementary rewiring actions that are specifically chosen to help align the user with their desired state. The system then creates a presentation that is delivered to the user through the presenter component. This presentation incorporates three distinct elements: a sensory component that engages the user's senses, an identity component that relates to how the user sees themselves, and an emotional component that connects with their feelings. The presentation is generated from the first-person perspective of the user to promote cognitive and behavioral transformation.

The system includes a tracking mechanism that monitors the user's progress over time as they work toward their desired state. Interventions can be delivered to users through various media formats including audio, video, and immersive virtual reality experiences. The interface may collect current data from the user through categorized inputs, and the system can be enhanced with biometric sensors and behavioral pattern tracking capabilities.

These categorized inputs draw from psychological, emotional, and physical dimensions, gathering information from direct user inputs, biometric sensor readings, and behavioral patterns identified by the tracking system. The system maintains a database of evidence-based methodologies for behavioral change, and the presentations are specifically tailored to interact with users based on these proven methodologies. The evidence-based approaches include neuroplasticity principles, cognitive behavioral therapy techniques, and emotional regulation strategies. Users can specify their preferred delivery format through the interface, and the system selects the presentation format based on user preferences, the situational context, and specific engagement requirements.

The gap between the current and desired states can be quantified using a structured data schema, which in one embodiment comprises a JSON object that organizes the relevant data for efficient processing.

The method for creating this personalized content begins with providing an interface that guides users or their proxies to enter information about their context, current state, and desired state. The system processes this entered information to define the context, current state, and desired state, then calculates the gap between the current and desired states. Based on this gap analysis, the system selects both a core rewiring action and multiple complementary rewiring actions designed to help align the user with their desired outcome. The system generates presentations from the user's first-person perspective, incorporating sensory, identity, and emotional components specifically designed to promote cognitive and behavioral transformation.

These presentations are delivered to users while the system simultaneously tracks their progress over time toward their desired state. The information collected can include personal or professional problems, the user's vision and goals, the user's desired traits or qualities, the user's obstacles and emotional drivers, known or inferred action steps, and instructional or conceptual goals. The system has the capability to quantify the gap between current and desired states and references accepted evidence-based methodologies for behavioral change when generating presentations.

Information is gathered through categorized inputs that span psychological, emotional, and physical dimensions, drawing from user inputs, biometric sensors, and behavioral pattern analysis. The presentation format is selected based on user preferences, situational context, and engagement requirements, and can include formats such as audiobook-style mentoring, identity-aligned visualization, first-person perspective delivery, episodic memory simulations, and episodic knowledge simulations.

The interface uses intelligent prompting, reflection logic, and adaptive clarification techniques to capture data in a structured schema that is optimized for modeling the gap between current and desired states. Presentations can be based on preloaded goal blueprints, therapist notes, teaching objectives, and external knowledge or goal schemas. These knowledge and goal schemas are derived from various sources including books, articles, educational materials, and content authored by experts in relevant fields.

The gap analysis identifies various types of challenges including emotional friction points, identity-level misalignment, behavioral bottlenecks, conceptual misunderstandings, and knowledge gaps. The system works to elicit prompt conditioning, identity context, and emotional priors through the interface functionality. Various data elements including current state, desired state, current obstacles, context, and desired emotions are managed using JSON formatting for structured data handling.

The system identifies conceptual material that needs to be taught to users and delivers this educational content through multiple first-person simulations. Both explicit user feedback and inferred user engagement patterns are stored by the system, which maintains an evolving user profile that enables optimization of future presentations based on the stored feedback and engagement data. This continuous learning approach allows the system to become more effective over time in delivering personalized content that facilitates meaningful behavioral and emotional transformation for each individual user.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors'intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF DRAWINGS

A more complete understanding of the present invention may be derived by referring to the detailed description when considered in connection with the following illustrative figures. In the figures, like reference numbers refer to like elements or acts throughout the figures.

FIG. 1 shows an example layout for onboarding a user of the system and method for creating personalized content to facilitate a behavioral and emotional transformation in a user in accordance to one or more embodiments;

FIG. 2 shows an example layout for onboarding a user of the system and method for creating personalized content to facilitate a behavioral and emotional transformation in a user in accordance to one or more embodiments;

FIG. 3 shows an example layout of a 3-year vision for a user of the system and method for creating personalized content to facilitate a behavioral and emotional transformation in a user in accordance to one or more embodiments;

FIG. 4 shows an example layout of understanding the user's problem of the system and method for creating personalized content to facilitate a behavioral and emotional transformation in a user in accordance to one or more embodiments;

FIG. 5 shows an example layout of understanding the user's goals of the system and method for creating personalized content to facilitate a behavioral and emotional transformation in a user in accordance to one or more embodiments;

FIG. 6 shows an example layout of generating a session of the system and method for creating personalized content to facilitate a behavioral and emotional transformation in a user in accordance to one or more embodiments;

FIG. 7 shows an example layout providing an immersive video and image generation of the system and method for creating personalized content to facilitate a behavioral and emotional transformation in a user in accordance to one or more embodiments;

FIG. 8 shows an example layout of a virtual reality system and mobile device running a presentation in accordance to one or more embodiments;

FIG. 9 shows a defined input schema according to one or more embodiments;

FIG. 10 shows a visualization flow from user input to personalized episodic simulation and audio delivery according to one or more embodiments;

FIG. 11 shows a book summary flow from goal-driven intake to personalized cognitive map summary based on selected content according to one or more embodiments;

FIG. 12 shows a diagram view of a system architecture according to one or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

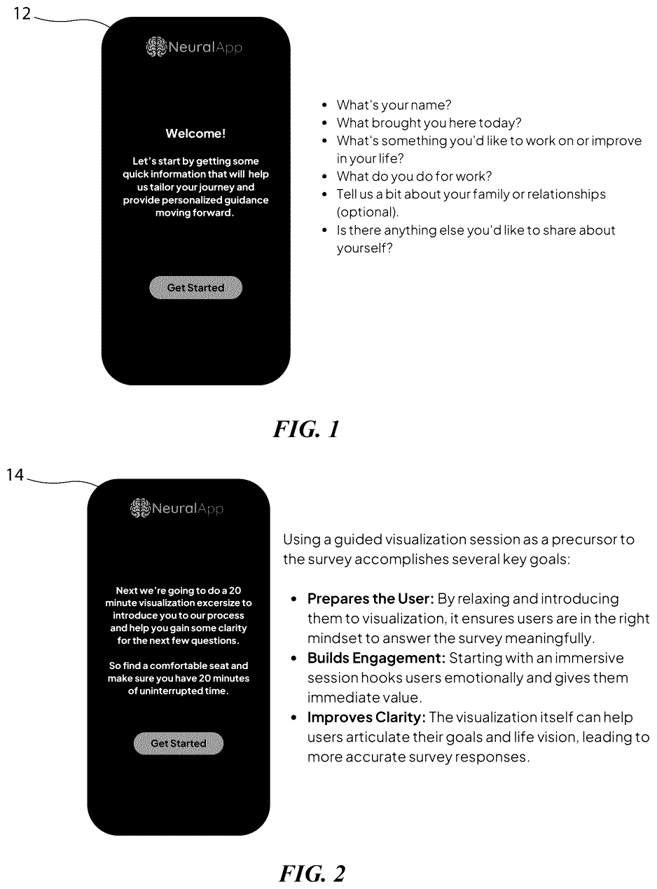

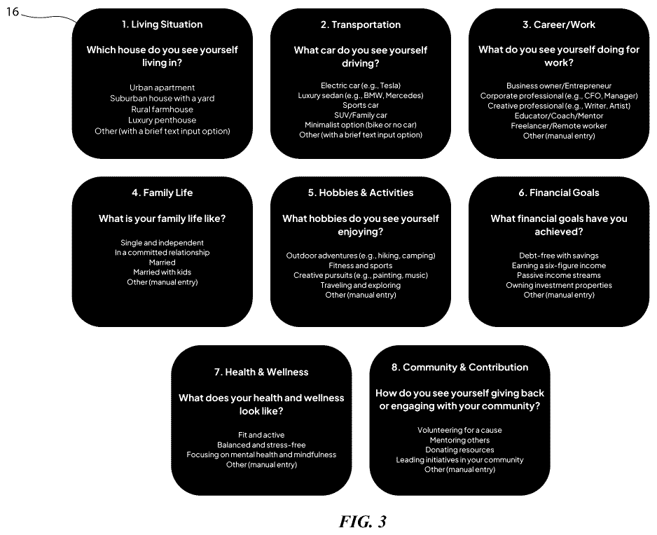

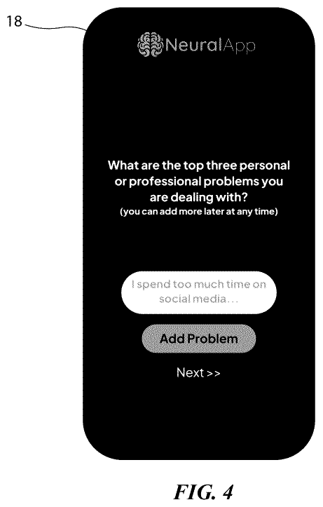

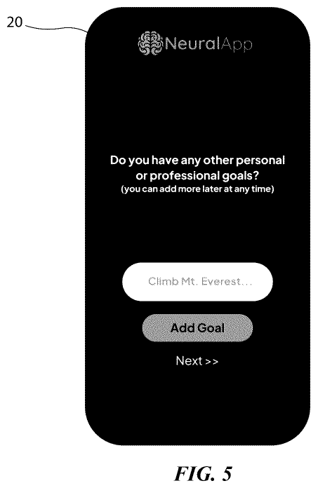

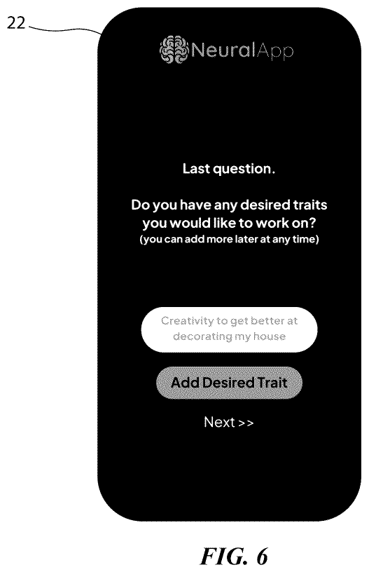

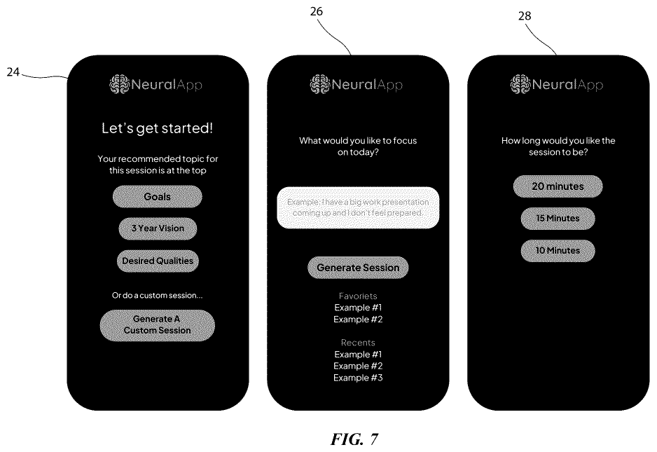

Referring initially to FIG. 1-6, and 12 a system and method for creating personalized content to facilitate behavioral and emotional transformation in a user is provided. The system aligns unconscious wiring with conscious intent using structured, emotionally charged mental simulations. Effective visualization can activate the same brain regions as real life experience, allowing users to rewire beliefs, habits, and identity through repetition, emotional immersion, and behavioral rehearsal.

The system can comprise several interconnected modules configured to collect user data, analyze the data, identify gaps between the user's current state and the user's desired state, generate tailored interventions, and deliver these interventions in engaging formats through audio, video and virtual reality (VR). During the user's onboarding process, a user state analyzer collects and analyzes data regarding a user's current state and the user's desired state. The user state analyzer can gather categorized inputs from the user, which can encompass psychological, emotional, and physical dimensions. These inputs can be collected from various sources, including self-reported data, biometric sensors, and observed behavioral patterns or as shown in FIGS. 1-6.

At 11, the user or user proxy is guided through a structured intake. At 12, the system asks the user questions such as, for example, name, what brought the user here today, something the user would like to improve on, what the user does for work, history of the user, substance abuse history, triggers, or the like. By integrating these diverse data streams and asking user specific questions, the user state analyzer module can create a comprehensive representation of the user's current state.

At step 14, the system guides users through a structured visualization session designed to prepare them mentally and emotionally before engaging with the survey questions. This preparatory session begins with relaxation techniques to help users release tension and achieve a calm, focused state of mind. Relaxation techniques may include deep breathing exercises, progressive muscle relaxation, or guided imagery. Fostering relaxation and clarity may reduce distractions, enhance cognitive readiness, and create a positive and reflective environment.

At step 16, the system provides a survey to the user to determine the user's three-year vision. The survey strategically structures questions to guide users through an introspective process, encouraging them to define clear, actionable goals. The survey asks the user about various life domains such as, for example: living situation (where they want to live, what type of home), transportation (how they want to get around, vehicles they want to own), career/work (what work they want to be doing), family life (relationships and family situations), hobbies and activities (what they want to spend time doing), financial goals (income and net worth targets), health and wellness (fitness and health objectives), community and contribution (how they want to impact others), and similar life areas.

The data collected from the survey feeds into the system's state analyzer module, which interprets the user's responses to identify patterns, priorities, and core motivations. The analysis may (1) summarize the user's vision in a clear and structured manner, (2) provide tailored guidance or feedback to align actions with goals, and (3) track progress toward these goals over time.

The system may generate a three-year summary in second-person, present tense format. For example: “In three years, you have [ownership details like home, car, business] and a net worth of [specific amount]. You [work details] for work. You spend time with [relationship details]. Day to day, you spend your time [time use activities].” This summary is presented to the user for confirmation before proceeding.

At step 18, the system prompts users to identify and articulate the top three personal or professional challenges they are currently facing. This serves as a starting point for targeted support and problem resolution. To assist users in pinpointing their most pressing concerns, the system may offer structured prompts and examples. Personal issues may include health struggles, stress management challenges, financial difficulties, or relationship conflicts. Professional challenges may include career stagnation, workload stress, leadership struggles, or skill gaps.

Users are given the flexibility to enter their challenges in their own words, enabling them to express both the scope and emotional weight of their issues. Optional follow-up prompts, such as “How long have you been dealing with this problem?” or “What would resolving this issue look like for you?” help users reflect on the nature and impact of these challenges.

Once the user has identified their top three challenges, the system guides them to prioritize based on urgency, impact, or feasibility of resolution. The input is analyzed by the system's internal state analyzer, which categorizes challenges into themes such as health, finance, career, relationships, or productivity. The internal state analyzer may identify root causes and highlight areas where solutions would be most impactful. For example, if “workload stress” is a user's concern, the system may suggest time management techniques or delegation strategies. Similarly, financial struggles might prompt recommendations such as budgeting tools or debt management resources.

At step 20, the system asks about the user's personal or professional goals to gain deeper insight into their motivations, aspirations, and preferences. Targeted questions may be used to understand the user's primary focus and tailor recommendations accordingly.

For instance, if the user identifies professional goals such as securing a leadership position, the system can tailor recommendations for relevant skill-building resources, leadership training programs, mentorship opportunities, or similar support. Similarly, if the user highlights personal goals like achieving physical fitness or mastering a creative hobby, the system can suggest personalized strategies, tools, and schedules to help them achieve these milestones.

At step 22, the system asks for traits the user may wish to target. The system may encourage the user to identify specific character traits or qualities they wish to improve, such as communication skills, confidence, resilience, time management, emotional intelligence, or similar attributes.

For instance, if a user expresses a desire to enhance their patience, the system can recommend strategies like mindfulness exercises (practices that focus attention on the present moment), techniques for managing stress, or practical scenarios to practice delayed gratification. Similarly, for users focused on improving leadership qualities, the system can suggest tailored activities such as team-building exercises, conflict resolution scenarios, or opportunities to lead small projects.

The system may take the user's input and add additional data such as engagement history (records of how the user has interacted with the system over time), behavioral data (patterns in the user's actions and choices), and sensor input (data from biometric devices) to supplement the user intake data. Based on the inputs from the user or a user proxy, the system defines: (1) the user's current state, (2) their desired future state, (3) obstacles and emotional drivers, (4) known or inferred action steps, and (5) appropriate instructional and conceptual goals.

Through a combination of intelligent prompting, reflection logic, automated assessments, diagnostic tools, and/or performance-based metrics, the user's current state is determined. User-specific schema extensions for particular scenarios such as addiction recovery, education, or other contexts may be used to further refine the process.

The system may guide the user through an iterative process to co-create and validate goals, ignition points (moments or factors that motivate action), actions, and sensory elements. The system can interact with the user using multiple-choice prompting (A/B/C selection) and user editing prior to simulation generation. In some embodiments, a proxy such as an educator or therapist may define learning objectives.

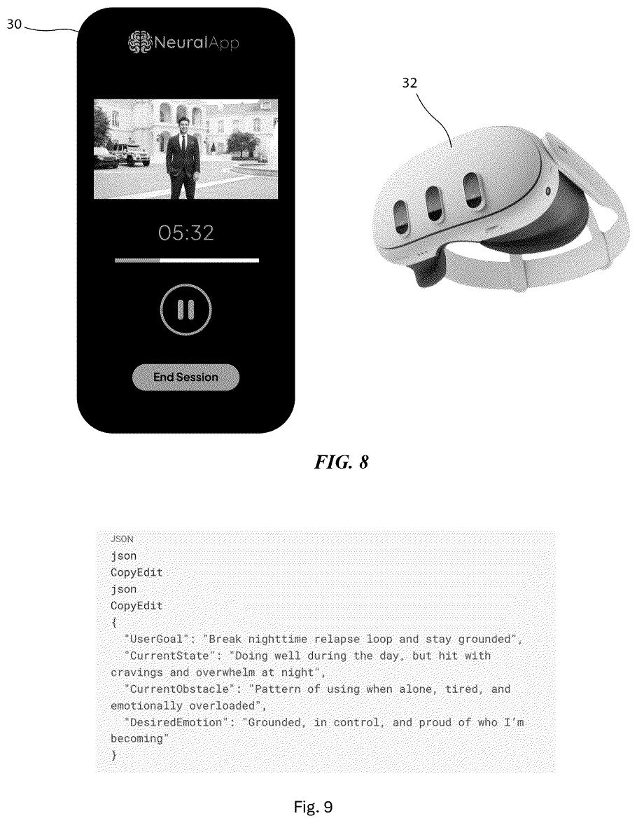

As shown in FIG. 9, the system may summarize the input in a defined input schema (a structured data format) that includes: (1) a user goal, (2) a current state, (3) a current obstacle, (4) a chosen book or other reference material (optional content sources that may supplement or shape the simulation), and (5) a desired emotion. This schema provides a standardized way to organize the information about what the user wants to achieve, where they currently are, what is blocking them, what resources they want to draw from, and how they want to feel.

At step 15, an artificial intelligence (AI) engine is employed to process the data collected by the user state analyzer module. The AI engine identifies and quantifies at least one gap between the user's current state and their desired state. A gap is the difference or distance between where someone is now and where they want to be.

The gap may include: (1) emotional friction points (situations where negative emotions create resistance to change), (2) identity-level misalignment (when how someone sees themselves doesn't match who they want to become), (3) behavioral bottlenecks (specific behaviors that prevent progress), (4) conceptual misunderstandings, or (5) knowledge gaps.

The analysis identifies gaps between the current and desired states, enabling the creation of targeted strategies to bridge these gaps. Gaps may be comprised of: (1) a motivational gap (the goal feels abstract, emotionally distant, or offers low reward), (2) a cognitive gap (the path forward is fragmented, unclear, or conceptually shallow), (3) an emotional gap (fear, doubt, or overwhelm disrupt engagement), and/or (4) a behavioral gap (desired actions feel unnatural or require significant effort).

The gap can be quantified through statistical and machine learning methods, which assign numerical values or qualitative descriptions to the discrepancies. Methods for quantifying the data can include similarity scores, deviation metrics, trend analyses, or similar analytical techniques. Deviation metrics measure the extent of the gap, such as the distance between actual and target attributes in a multidimensional space.

The system prioritizes traits based on their connection to the user's defined goals, challenges, and vision for the future. The system transforms user inputs into actionable strategies, delivering a personalized experience that aligns with individual goals and aspirations. Intervention pathways may be user-validated before being implemented, meaning users can review and approve the recommended approaches before the system proceeds.

At step 19, using a content generation module, the system dynamically creates personalized interventions for the user that are aimed at bridging the user's identified gaps. These interventions are grounded in evidence-based principles such as: (1) neuroplasticity to support cognitive and behavioral transformation, (2) cognitive behavioral therapy (CBT), and (3) emotional regulation techniques. The interventions are tailored to the user's unique profile, preferences, and progress over time to provide high relevance and effectiveness.

The content generation module employs advanced algorithms to analyze the specific nature of the gaps, which include psychological, emotional, or physical dimensions, and generates targeted content designed to facilitate the desired transformation. Advanced algorithms can include: natural language processing (NLP), predictive analytics, clustering and segmentation, neural networks, recommendation and optimization algorithms, decision trees and ensemble models, knowledge graphs and semantic analysis, or similar computational techniques.

The content generation module may use any combination of evidence-based therapeutic principles and advanced computational techniques to provide interventions that are both effective and personalized. The therapeutic principles include: neuroplasticity to promote cognitive and behavioral change by reinforcing or creating new neural pathways, cognitive behavioral therapy (CBT) to address maladaptive thought patterns and replace them with constructive ones, and emotional regulation strategies to enhance the user's ability to manage and stabilize their emotional states.

The content generation module is further configured to adapt its output dynamically based on user-specific inputs and progress. For example, it may analyze real-time feedback from the user, including engagement levels, biometric responses, and self-reported satisfaction, to adjust the content for optimal effectiveness. The interventions can also evolve over time, increasing in complexity or intensity as the user demonstrates progress or readiness for more advanced exercises.

The simulation generator is model-agnostic, meaning it can work with any AI engine capable of structured semantic generation, identity context processing, and emotionally relevant content synthesis. This includes, but is not limited to: transformer-based large language models (LLMs), retrieval-augmented generation systems (RAG), fine-tuned smaller models, embedded emotion-aware networks or multimodal models, or on-device or serverless model deployments. Logical layers may be decoupled from model vendors, allowing substitution, upgrades, or privacy-preserving deployments without loss of core function.

Content may include: audiobook-style mentoring, identity-aligned visualization, first-person episodic memory simulations, or episodic knowledge simulations for internalizing strategies, models, or conceptual frameworks.

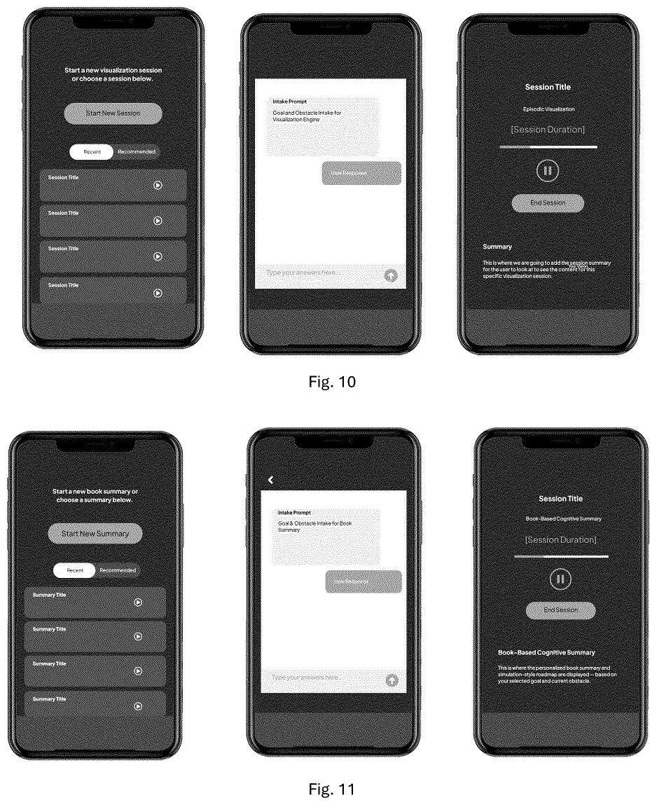

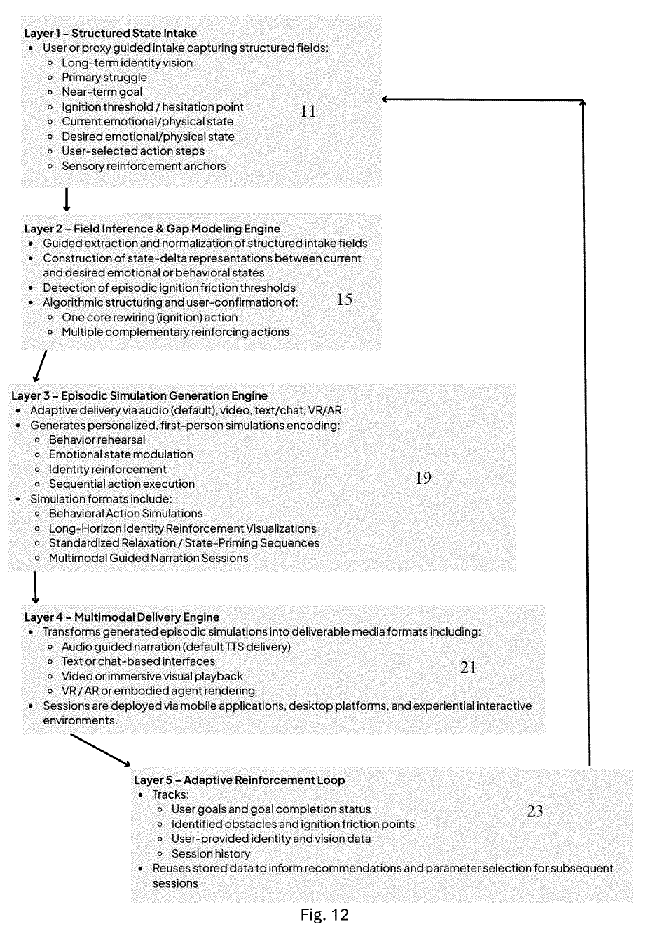

As shown in FIG. 10, the visualization flow moves from user input through to personalized episodic simulation and audio delivery. The simulation structure targets specific neural systems including the premotor cortex, insula, hippocampus, and ventromedial prefrontal cortex.

Delivered sessions activate the hippocampus, premotor cortex, and insula by generating vivid, first-person simulations of future actions and outcomes. This mimics the brain's use of episodic future thinking (the ability to imagine future events as if experiencing them) to prepare for real-world behavior. Structured content such as instructional material or book-based lessons may be transformed into episodic-style simulations of emotionally resonant action-outcome sequences aligned to the user's internal state, context, and goal.

These simulations are structured as cognitive maps. A cognitive map is a mental representation of how to navigate from one state to another, including: intent, struggle, insight (understanding what needs to be done), and reward. This structure helps users mentally rehearse the entire journey from where they are to where they want to be.

Sessions follow a cue→delta→action→reward→identity sequence. A “cue” is a trigger or situation that prompts behavior. “Delta” refers to the change or gap that needs to be addressed. “Action” is the specific behavior or step taken. “Reward” is the positive outcome experienced. “Identity” is the updated self-concept that results from consistent action and reward. This sequence mirrors how the brain naturally forms and reinforces habits.

The short-term session follows a four-phase flow: cue, state, action, and reward. This mirrors the brain's habit loop. Users imagine a setting (cue), sense their current state, take action, and experience reward. This sequence activates the same motor, emotional, and memory circuits as real performance. Repeated exposure consolidates the pattern through reconsolidation (the process by which memories are strengthened each time they are recalled) and prediction-error learning (learning that occurs when outcomes differ from expectations), turning intention into embodied readiness.

The long-term vision module provides a persistent identity framework that feeds into each short-term session. This framework includes information about ownership, net worth, work, relationships, and time use. This aligns immediate goals with the user's long-term identity schema and self-concept.

The long-term vision module re-activates the reward state and extends it to life contexts like work, ownership, relationships, time use, and net worth. It bridges short-term episodic learning with long-term self-schema formation in the medial prefrontal cortex. Script variants target different memory integration channels. These variants may include sensory simulations (focusing on physical sensations), procedural simulations (focusing on step-by-step actions), relational simulations (focusing on interactions with others), and narrative simulations (focusing on the story of transformation). Each variant stabilizes the user's future identity as real and familiar.

Together, the short-term and long-term modules create a closed neurobehavioral loop: short-term sessions encode action as memory, and long-term sessions integrate memory as identity. This continuous reinforcement builds emotional alignment, behavioral consistency, and durable self-belief.

At step 24, the system provides users with the option to either select a recommended topic for their session or create a fully customized session tailored to their unique needs. The recommended topics are curated based on the user's previous interactions, preferences, or identified goals to provide relevance and alignment with their current priorities. Alternatively, the system allows users to design their own session by specifying a subject or focus area that resonates with their immediate needs, fostering greater personalization and engagement.

As shown in FIG. 11, the system can also implement a book summary flow from goal-driven intake to personalized cognitive map summary based on selected content. Users can select books, articles, educational materials, or expert-authored content as the basis for their learning sessions. The system transforms this content into episodic simulations that help users internalize and apply the concepts.

Optional content sources may also be used to supplement or shape the simulations. Users can benefit from structured frameworks like root cause analysis, SMART goal-setting methods (Specific, Measurable, Achievable, Relevant, Time-bound goals), step-by-step action plans, or other content from therapist/teacher notes, books, articles, educational materials, expert-authored content, or preloaded goal blueprints that are personalized to user needs.

At step 26, the system prompts the user to define their primary focus for the session. At step 28, the user is given the flexibility to determine the duration of the session. The system can offer preset durations such as 5, 10, 20, or 30 minutes, or allow the user to specify a custom length, enhancing adaptability and convenience.

At step 21, and also referring to FIG. 8, at steps 30 and 32, a delivery module is configured to present the personalized interventions to the user through at least one of audio, video, textual narrative, or immersive virtual reality (VR) formats. The delivery format is selected based on factors such as the user's preferences, situational context, and engagement requirements to provide interventions that are accessible, engaging, and impactful. The delivery module is configured to present personalized interventions to the user through a variety of formats, including audio, video, and immersive virtual reality (VR) to provide versatility and adaptability to meet individual user needs.

Interventions may have one or more narrative structures including somatic, procedural, relational, and narrative structures. The module selects the most suitable delivery format based on a range of factors, such as the user's preferences, situational context, engagement requirements, and the specific objectives of the intervention. The dynamic selection process provides the interventions remain accessible, engaging, and impactful.

The delivery module tailors the content presentation to maximize the user's ability to absorb and interact with the intervention. Content may be delivered by at least one of: audio, video, virtual reality, augmented reality, robotic interfaces, textual interfaces, haptic feedback, olfactory stimulation, or other sensory channels.

For audio delivery, the module generates and delivers guided meditations, cognitive exercises, or affirmations that are presented through headphones or speakers. Audio interventions may include voice modulation, and ambient sounds to enhance the immersive quality and emotional impact.

For video delivery, the module creates and presents visual content, such as instructional tutorials, emotionally engaging scenarios, animated guides, or similar content that demonstrates specific techniques or exercises. Video content can be streamed or downloaded and is often supplemented with captions, annotations, or interactive elements to enhance user understanding and engagement.

The delivery module can also provide immersive VR interventions, which offer high engagement by creating fully interactive and realistic environments. VR content may include simulated scenarios for exposure therapy, interactive environments for behavioral activation, or calming and meditative spaces designed to reduce stress and enhance focus. The module can leverage VR hardware, such as headsets and controllers, to facilitate an immersive experience that enables the user to actively participate in the intervention.

The module has real-time adaptability to provide effective delivery. For example, it can adjust the pacing, tone, or complexity of the intervention based on user engagement metrics, such as attention span, biometric feedback, or progress data. If the user exhibits signs of stress or disengagement, the module may modify the intervention to provide a more suitable experience, such as switching from an intense VR scenario to a calming audio meditation.

To further personalize the experience, the delivery module integrates contextual awareness. It considers factors such as the user's environment (for example, noise levels, available equipment), time of day, and activity level to optimize content delivery. For instance, it may prioritize audio-based interventions during a user's commute or suggest VR experiences in a quiet, private setting.

The module facilitates seamless interaction with the user. It can provide notifications or prompts to encourage participation, offer guidance on using the required hardware, and track user feedback to refine future interventions. By providing personalized interventions are delivered in the most effective and user-friendly format, the delivery module bridges the gap between the user's current state and user's desired state.

At step 23, to provide ongoing improvement, the system tracks the user's progress, allowing for periodic updates, monitoring trends, and adapting strategies as new challenges arise. The system can integrate external tools, such as fitness applications, task management platforms, health data from wearables, or similar sources that can infer progress indirectly to the system.

This inquiry not only enhances the system's ability to provide relevant and meaningful guidance but also fosters user engagement by showing responsiveness to their individual needs. Furthermore, by analyzing trends in the user's responses over time, the system can identify changes or shifts in priorities and adapt its recommendations accordingly. For example, if the user initially focuses on professional goals but later prioritizes health and wellness, the system can adjust its approach to align with the user's evolving circumstances.

In embodiments, the method can adjust the content dynamically based on user engagement and feedback, tailoring the delivery format and intensity to optimize outcomes. For example, VR scenarios can adapt to the user's physiological responses, while audio and video sessions can scale in complexity as the user demonstrates progress.

The system stores explicit user feedback and inferred user engagement patterns, maintaining an evolving user profile that enables optimization of future presentations based on the stored feedback and engagement data. Users mark completed goals in the system with a checkmark or similar indicator to trigger a micro-reward: immediate visual and emotional feedback, dashboard updates, and re-activation of effort and accomplishment reward circuits in the brain.

The system is content-agnostic, meaning it applies across education, therapy, training, performance, or leadership without altering its neuroscience foundation. It provides a universal framework for encoding goal-directed behavior through emotionally grounded, sensory simulation that links user visions with brain responses. The system applies broadly across behavior-change and learning verticals, including: addiction recovery, education, therapy, leadership development, and performance optimization.

In an example embodiment, a user identifies public speaking as a significant challenge, expressing a desire to become a confident speaker while acknowledging procrastination as a recurring issue, particularly when preparing for presentations. The user aspires to cultivate traits such as “confidence” and “discipline” to overcome these barriers and achieve success in professional settings.

The system processes the user's inputs by categorizing them into a structured framework. It identifies the user's current state as experiencing anxiety about public speaking and a tendency to procrastinate, while their desired state involves delivering impactful presentations with confidence and adhering to a structured preparation schedule. The overarching goal may be identified to be developing the confidence and discipline needed to excel in public speaking while minimizing procrastination. Personalized interventions are dynamically generated to replace these avoidance patterns with proactive behaviors, such as practicing presentation skills, and to reduce anxiety through repeated positive associations with successful public speaking experiences.

The system uses a combination of affirmations, visualizations, and neural exercises to target confidence-building and discipline while addressing underlying anxiety. Affirmations such as “Each time I practice, I grow more confident and prepared” and “I deliver my message with clarity, passion, and impact” help instill a positive mindset.

Guided visualizations lead the user through scenarios where they deliver presentations to supportive audiences, feeling calm, prepared, and confident. Visualizations may also depict breaking down the preparation process into manageable steps, reinforcing discipline and reducing the overwhelm associated with procrastination.

The neural exercises guide the user through progressive exposure to public speaking scenarios, starting with low-stakes situations and gradually building to more challenging ones. Simulated time management strategies may be provided, associating structured preparation with positive outcomes like reduced anxiety and successful delivery.

As another example, the system can help a user who has set a goal to complete their first marathon but struggles with maintaining motivation and adhering to a structured training plan, particularly on days when they feel tired or unmotivated. The user identifies the need to develop traits such as “discipline” and “perseverance” to overcome these challenges and achieve their goal.

The system processes the user's input by categorizing their current state as inconsistent training due to low motivation and their desired state as one where they demonstrate the discipline and perseverance required to stick to their schedule and complete the marathon. The overarching goal is to build the mental and emotional resilience needed to achieve marathon readiness and successfully finish the race.

To address these challenges, the system focuses on neural rewiring to transform avoidance behaviors caused by fatigue or lack of motivation into proactive responses. Interventions dynamically generated by the system associate these challenges with opportunities for growth and achievement. Tailored affirmations such as “Each run makes me stronger and more prepared for the challenge ahead” and “I embrace every step of my training journey, knowing it brings me closer to my goal” help reinforce positive attitudes toward training.

Guided visualizations immerse the user in scenarios where they overcome fatigue to complete training sessions and experience the pride that follows. Another visualization places the user at the marathon's starting line, feeling confident and ready, culminating in vivid imagery of crossing the finish line surrounded by cheering supporters.

Neural exercises, including resilience drills may be provided. Through these tailored affirmations, visualizations, and neural exercises, the system empowers the user to build discipline, persevere through challenges, and ultimately achieve their marathon goal in a personalized and effective manner.

In another example, the system assists a user who sets a goal to achieve sobriety, identifying challenges such as stress-induced cravings and feelings of inadequacy in social settings. The user seeks to cultivate traits like “emotional resilience” and “self-worth” to lead a fulfilling, substance-free life. The system processes the user's input by categorizing their current state as experiencing stress and social discomfort that trigger cravings for alcohol, and their desired state as one of sobriety accompanied by confidence and self-worth in social interactions. The overarching goal is to develop resilience to stress and reprogram automatic responses to cravings.

To address these challenges, the system focuses on neural rewiring to transform maladaptive pathways where stress triggers cravings into healthier associations, such as stress prompting positive behaviors like relaxation techniques or social confidence. Affirmations such as “Each time I choose a positive action, I strengthen new, empowering pathways in my brain,” “I deserve a life of clarity, confidence, and freedom,” and “Stress is an opportunity for growth, and I respond with strength and calm” are generated to reinforce these new behaviors.

Guided visualizations immerse the user in scenarios where they respond to stressful events without alcohol, maintaining a sense of calm and control. These visualizations also depict the long-term benefits of sobriety, such as improved relationships, better health, and personal achievements, providing motivational reinforcement. Neural exercises involving gradual exposure to stressors through mental rehearsals may be provided. These exercises reinforce alternative responses that engage the prefrontal cortex for decision-making and deactivate the amygdala to reduce emotional reactivity. For example, the user is guided through a visualization of a social event where they confidently decline alcohol and feel fulfilled by meaningful interactions.

At least one of the current state, the desired state, a current obstacle, a context, and a desired emotion may be managed using JavaScript Object Notation (JSON). This standardized format enables consistent data handling and processing across the system.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

I claim:

1. A system for creating personalized content to facilitate a behavioral and/or emotional transformation in a user, the system comprising:
an interface including an input and a presenter;
at least one memory storing instructions that, when executed by at least one processor, configure the interface to:
guide the user or a user proxy to enter information;
define a context and a desired state based on the entered information; and
select a core rewiring action and a plurality of complementary rewiring actions designed to align the user with the desired state; and
wherein the presenter presents at least one presentation to the user and the presentation comprises a sensory component, an identity component, and an emotional component.

2. The system of claim 1, further comprising a tracker to track the user's progress over time around the user's desired state.

3. The system of claim 1, wherein the interface collects current data from categorized inputs from the user.

4. The system of claim 2, further comprising at least one biometric sensor and a behavioral pattern tracker, wherein the categorized inputs are taken from at least one of a psychological, an emotional, and/or a physical dimension taken from at least one of the user's inputs, the biometric sensors, and behavioral patterns from the behavioral pattern tracker.

5. The system of claim 1, wherein the interface provides a form for the user to enter a delivery format preference and wherein the presentation has a format that is selected based on at least one of the user's preferences, situational context, and engagement requirements.

6. The system of claim 1 wherein the context and desired state are entered using a structured data schema with a JSON object.

7. A method for creating personalized content to facilitate a behavioral and/or emotional transformation in a user, the method comprising:
providing an interface configured to guide the user or a user proxy to enter information concerning a context and a desired state of the user;
defining the context and the desired state based on the entered information;
selecting a core rewiring action and a plurality of complementary rewiring actions designed to align the user with the desired state;
generating at least one presentation from the first-person perspective of the user wherein the presentation comprises a sensory component, an identity component, and an emotional component to promote cognitive and behavior transformation; and
delivering the at least one presentation to the user.

8. The method of claim 7, wherein the information is at least one of a personal or professional problem, the user's vision, the user's goals, and the user's desired traits or qualities.

9. The method of claim 7, further comprising defining a current state and a gap between the current state and the desired state.

10. The method of claim 9, wherein the gap is identified using a structured data schema with a JSON object.

11. The method of claim 7, wherein the information is collected from categorized inputs from the user.

12. The method of claim 11, wherein the categorized inputs are taken from at least one of a psychological, an emotional, and/or a physical dimension taken from the user's inputs, biometric sensors, and behavioral patterns.

13. The method of claim 7, wherein the presentation has a format selected based on at least one of the user's preferences, situational context, and engagement requirements.

14. The method of claim 13 wherein the format is at least one of audiobook style mentoring, identity-aligned visualization, first-person, episodic memory simulations, and episodic knowledge simulations.

15. The method of claim 7, wherein the information includes the user's obstacles and emotional drivers, known or inferred action steps, and instructional or conceptual goals.

16. The method of claim 9, wherein the interface accepts input using at least one of intelligent prompting, reflection logic, and adaptive clarification to capture data in a structured schema optimized for modeling the gap.

17. The method of claim 7, wherein the at least one presentation is based on at least one of preloaded goal blueprints, therapist notes, teaching objectives, and external knowledge or goal schemas.

18. The method of claim 9, wherein the gap identifies at least one of emotional friction points, identity-level misalignment, behavioral bottlenecks, conceptual misunderstandings, and knowledge gaps.

19. The method of claim 7, further comprising eliciting at least one of prompt conditioning, identity context, emotional priors through the interface.

20. The method of claim 7, wherein at least one of a current state, the desired state, a current obstacle, a context, and a desired emotion are managed using a JSON format.

21. The method of claim 7, further comprising identifying conceptual material to be taught to the user and teaching the conceptual materials through a plurality of first-person simulations.

22. The method of claim 7, further comprising storing at least one of explicit user feedback and inferred user engagement patterns and maintaining an evolving user profile to optimize future presentations based on the stored explicit user feedback or inferred user engagement patterns.
